(19) United States
(12) Patent Application Publication
(10) Pub. No.: US 2024/0376309 A1
(43) Pub. Date: Nov. 14, 2024

(54) COPOLYESTER BLENDS

(71) Applicant: Eastman Chemical Company, Kingsport, TN (US)

(72) Inventors: Joshua Seth Cannon, Greeneville, TN (US); Scott Ellery George, Kingsport, TN (US); Coralie McKenna Fleenor, Rogersville, TN (US); Huamin Hu, Johnson City, TN (US); Mark Allan Treece, Jonesborough, TN (US); Carolin Adleheid Vogel, Kingsport, TN (US); Matthew Robert Kita, Johnson City, TN (US); Jason Scott Woods, Kingsport, TN (US); Jonathan Michael Horton, Kingsport, TN (US)

(73) Assignee: Eastman Chemical Company, Kingsport, TN (US)

(21) Appl. No.: 18/688,090

(22) PCT Filed: Aug. 25, 2022
(86) PCT No.: PCT/US2022/041479

§ 371 Date: Feb. 29, 2024

Related U.S. Application Data
(60) Provisional application No. 63/260,752, filed on Aug. 31, 2021.
(60) Provisional application No. 63/260,753, filed on Aug. 31, 2021.
(60) Provisional application No. 63/260,755, filed on Aug. 31, 2021.
(60) Provisional application No. 63/260,756, filed on Aug. 31, 2021.
(60) Provisional application No. 63/260,758, filed on Aug. 31, 2021.
(60) Provisional application No. 63/260,761, filed on Aug. 31, 2021.
(60) Provisional application No. 63/260,758, filed on Aug. 31, 2021.

(51) Int. Cl. C08L 67/02; C08G 63/199; C08G 63/672; C08G 63/86

(52) U.S. Cl. CPC C08L 67/02; C08G 63/199; C08G 63/672; C08G 63/863

(57) ABSTRACT

A copolyester composition is provided comprising at least one copolyester and at least one polymeric component; wherein said copolyester comprises: a. terephthalate acid residues; b. about 85 to about 96 mole % of ethylene glycol residues; c. about 4 to about 15 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD); and d. a germanium catalyst present in the copolyester at a concentration of about 5 to about 500 ppm based on elemental germanium; wherein the terephthalate monomer is based on the substantially equal diacid equivalents of 100 mole % to diol equivalence of 100 mole % for a total of 200 mole %.

### FIELD OF THE INVENTION

The present invention relates to copolyesters using germanium catalyst. More specifically, the invention relates to copolyesters using germanium catalyst wherein the copolyesters provide excellent color with a low diethylene glycol content. Processes for producing these copolyesters are also provided as well as articles comprising the inventive copolyesters. More particularly, the inventive copolyesters are useful for molding thick wall parts, such as jars that are compatible with the PET recycle stream.

### BACKGROUND OF THE INVENTION

There is a need for copolyesters with excellent appearance and long crystallization half-times to allow for the fabrication of thick wall containers. The counterbalance to solving this problem is that growing environmental concerns require compatibility with the RIC1 stream as specified by a crystalline melting point ≥225° C. Titanium and antimony catalysts, typically used for synthesis of PET are not suitable since titanium results in higher yellowness and a darker polymer when toner dyes are added. In addition, antimony tends to reduce to Sb(0) in the presence of 1,4-cyclohexanedimethanol (CHDM) or 2,2,4,4-tetramethyl-1,3-cyclobutane diol (TMCD) resulting in a hazy/grayish appearance.

This problem is solved by using a germanium catalyst to provide a copolyester, particularly polyethylene terephthalate (PET), that is modified with a total of 15 mole % or less of a diethylene glycol comonomer and at least one glycol comonomer selected from the group consisting of 1,4-cyclohexanedimethanol (CHDM), monopropylene glycol (MPG), and 2,2,4,4-tetramethyl-1,3-cyclobutane diol (TMCD). Another aspect of this invention is that it is not necessary to minimize the level of DEG to decrease crystallization half-time and maintain excellent color when germanium alone is used as the polycondensation catalyst.

### BRIEF SUMMARY OF THE INVENTION

In one embodiment of the invention, a copolyester is provided comprising: a) terephthalate acid residues; b) about 85 to about 96 mole % of ethylene glycol residues; c) about 4 to about 15 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD); and d) a germanium catalyst present in the copolyester at a concentration of about 5 to about 500 ppm based on elemental germanium; wherein the terephthalate monomer is based on the substantially equal diacid equivalents of 100 mole % to diol equivalence of 100 mole % for a total of 200 mole %.

In another embodiment of the invention, a copolyester is provided comprising a) terephthalate acid residues, b) about 85 to about 96 mole % of ethylene glycol, c) about 4 to about 15 mole % of a combination of 1,4-cyclohexanedimethanol (CHDM) and diethylene glycol (DEG), and d) a germanium catalyst present in the copolyester at a concentration of about 5 to about 500 ppm based on elemental germanium; wherein the diacid is based on the substantially equal diacid equivalents of 100 mole % to diol equivalence of 100 mole % for a total of 200 mole %.

In another embodiment of the invention, a copolyester composition is provided comprising at least one copolyester and at least one polymeric component; wherein said copolyester comprises:
a. terephthalate acid residues;
b. about 85 to about 96 mole % of ethylene glycol residues;
c. about 4 to about 15 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD); and
d. a germanium catalyst present in the copolyester at a concentration of about 5 to about 500 ppm based on elemental germanium; wherein the terephthalate monomer is based on the substantially equal diacid equivalents of 100 mole % to diol equivalence of 100 mole % for a total of 200 mole %.

In another embodiment of the invention, a process to produce a copolyester is provided comprising:
a. polymerizing
   1) at least one terephthalate monomer;
   2) about 85 to about 96 mole % of ethylene glycol;
   3) about 4 to about 15 mole % of a combination diethylene glycol (DEG) and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD);
in the presence of a germanium catalyst to produce said copolyester; wherein said germanium catalyst is present in said copolyester at a concentration of about 5 to about 500 ppm based on elemental germanium; wherein the diacid is based on the substantially equal diacid equivalents of 100 mole % to diol equivalence of 100 mole % for a total.

In another embodiment of the invention, an article is provided comprising a copolyester; wherein said copolyester comprises:
a. at least one terephthalate monomer residue;
b. about 85 to about 96 mole % of ethylene glycol residues;
c. about 4 to about 15 mole % of a combination diethylene glycol (DEG) and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD);
d. a germanium catalyst present in the copolyester at a concentration of about 5 to about 500 ppm based on elemental germanium; wherein the diacid monomer is based on the substantially equal diacid equivalents of 100 mole % to diol equivalence of 100 mole % for a total of 200 mole %.

### BRIEF DESCRIPTION OF THE DRAWINGS

FIG. 1—Copolyesters similar in molecular weight with about a 12 mole % total glycol modification were obtained using the procedures described in Examples 1-4 and the results are provided in FIG. 1.

FIG. 2—Copolyesters similar in molecular weight with about a 10 mole % total glycol modification were obtained using the procedures described in Examples 1-4 and the results are provided in FIG. 2.

### DETAILED DESCRIPTION OF THE INVENTION

The present invention may be understood more readily by reference to the following detailed description of certain embodiments of the invention and the working examples. In accordance with the purpose(s) of this invention, certain embodiments of the invention are described in the Summary of the Invention and are further described herein below. Also, other embodiments of the invention are described herein.

### Copolyester

In one embodiment of the invention, a copolyester is provided comprising:
a. terephthalate acid residues;
b. about 85 to about 96 mole % of ethylene glycol residues;
c. about 4 to about 15 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD); and
d. a germanium catalyst present in the copolyester at a concentration of about 5 to about 500 ppm based on elemental germanium; wherein the terephthalate monomer is based on the substantially equal diacid equivalents of 100 mole % to diol equivalence of 100 mole % for a total of 200 mole %.

In another embodiment of the invention, a copolyester is provided comprising:
a. terephthalate acid residues;
b. about 85 to about 96 mole % of ethylene glycol residues;
c. about 4 to about 15 mole % of a combination of 1,4-cyclohexanedimethanol (CHDM) and diethylene glycol residues (DEG); and
d. a germanium catalyst present in the copolyester at a concentration of about 5 to about 500 ppm based on elemental germanium; wherein the diacid monomer is based on the substantially equal diacid equivalents of 100 mole % to diol equivalence of 100 mole % for a total of 200 mole %.

The present invention relates to copolyesters produced using germanium as the polycondensation catalyst to synthesize a copolyester comprised of terephthalate acid residues, ethylene glycol residues, diethylene glycol residues, and at least one glycol residue selected from the group consisting 1,4-CHDM residues, MPG residues, and TMCD residues.

It is believed that the inventive copolyesters provide at least one of the following unique properties: 1) crystallization half times of greater than one minute; 2) melting temperature of equal to or greater than 225° C. which allows for R1C1 recycling; 3) an inherent viscosity of at least 0.2; and 4) little or no haze when extrusion blow molded;

The term “polyester,” as used herein, is intended to include “copolyesters” and is understood to mean a synthetic polymer prepared by the reaction of one or more difunctional carboxylic acids and/or multifunctional carboxylic acids with one or more difunctional hydroxyl compounds and/or multifunctional hydroxyl compounds. Typically the difunctional carboxylic acid can be a dicarboxylic acid and the difunctional hydroxyl compound can be a dihydric alcohol such as, for example, glycols and diols.

The term “glycol” as used herein includes, but is not limited to, diols, glycols, and/or multifunctional hydroxyl compounds, for example, branching agents.

As used herein, the term “dicarboxylic acid” is intended to include dicarboxylic acids as well as multifunctional carboxylic acids and any derivative of a dicarboxylic acid or multifunctional carboxylic acid, for example, branching agents. The term “dicarboxylic acid” also includes the associated acid halides, esters, half-esters, salts, half-salts, anhydrides, mixed anhydrides, and/or mixtures thereof, useful in a reaction process with a diol to make polyester. Alternatively, the difunctional carboxylic acid may be a hydroxy carboxylic acid such as, for example, p-hydroxybenzoic acid, and the difunctional hydroxyl compound may be an aromatic nucleus bearing 2 hydroxyl substituents such as, for example, hydroquinone.

The term “residue,” as used herein, means any organic structure incorporated into a polymer through a polycondensation and/or an esterification reaction from the corresponding monomer.

The term “repeating unit,” as used herein, means an organic structure having a dicarboxylic acid residue (acid residue) and a diol residue (glycol residue) bonded through a carbonyloxy group. Thus, for example, the term “dicarboxylic acid residues,” is used interchangeable with the term “acid residues,” and may be derived from a dicarboxylic acid monomer or its associated acid halides, esters, salts, anhydrides, and/or mixtures thereof.

As used herein, the term “terephthalic acid” is intended to include terephthalic acid itself and residues thereof as well as any derivative of terephthalic acid, including its associated acid halides, esters, half-esters, salts, half-salts, anhydrides, mixed anhydrides, and/or mixtures thereof or residues thereof useful in a reaction process with a diol to make polyester.

The polyesters used in the present invention typically can be prepared from dicarboxylic acids and glycols which react in substantially equal proportions and are incorporated into the polyester polymer as their corresponding residues. The polyesters of the present invention, therefore, can contain substantially equal molar proportions of acid residues (100 mole %) and glycol residues (100 mole %) such that the total moles of repeating units are equal to 100 mole %. The mole percentages provided in the present disclosure, therefore, may be based on the total moles of acid residues, the total moles of glycol residues, or the total moles of repeating units. For example, a polyester containing 10 mole % isophthalic acid, based on the total acid residues, means the polyester contains 10 mole % isophthalic acid residues out of a total of 100 mole % acid residues. Thus, there are 10 moles of isophthalic acid residues among every 100 moles of acid residues. In another example, a polyester containing 15 mole % 1,4-cyclohexanedimethanol out of a total of 100 mole % glycol residues has 15 moles of 1,4-cyclohexanedimethanol residues among every 100 moles of glycol residues. Also, for example, a polyester containing 0.5 mole % trimellitic anhydride residues contains 0.5 moles of trimellitic anhydride residues for every 100 moles of acid residues. Likewise, a polyester containing 0.5 mole % trimethylolpropane residues contains 0.5 moles of trimethylolpropane residues for every 100 moles of glycol residues.

As used herein, the term “branching agent” is equivalent to branching monomer and is a multifunctional compound with either hydroxyl or carboxyl substituents that can react with the difunctional monomers of the polyester. The term “multifunctional” refers to functional compounds that are not mono-functional or difunctional.

As used herein the term “extrusion blow molding process” has its usual meaning to one skilled in the art and includes any extrusion blow molding manufacturing process known in the art. Although not limited thereto, a typical description of extrusion blow molding manufacturing process involves: 1) melting the resin in an extruder; 2) extruding the molten resin through a die to form a tube of molten polymer (i.e. a parison); 3) clamping a mold having the desired finished shape around the parison; 4) blowing air into the parison, causing the extrudate to stretch and expand to fill the mold; 5) cooling the molded article; 6) ejecting the article from the mold; and 7) removing excess plastic (commonly referred to as flash) from the article. As used herein, the term “extrusion blow molded article” is any article made by an extrusion blow molding process including but not limited to a container, a bottle, or a through-handle bottle.

The term “container” as used herein is understood to mean a receptacle in which material is held or stored. Containers include but are not limited to bottles, bags, vials, tubes and jars. Applications in the industry for these types of containers include but are not limited to food, beverage, cosmetics, and personal care applications.

The term “bottle” as used herein is understood to mean a receptacle containing plastic which is capable of storing or holding liquid.

As used herein, the term “haze” is the ratio of diffuse transmittance to total light transmittance. Haze is measured on sidewalls of extrusion blow molded articles according to ASTM D 1003, Method A, and is calculated as a percentage. A BYK-Gardner HazeGuard Plus was used to measure haze.

As used herein the term “inherent viscosity” or “IhV” is the viscosity of a dilute solution of the polymer, specifically IhV is defined as the viscosity of a 60/40 (wt/wt) phenol/tetrachloroethane at a concentration of 0.25 g polyester per 50 ml solution at a specified temperature of either 25° C. or 30° C.

As used herein, the term “intrinsic viscosity” or “ItV” is the ratio of a solutions specific viscosity to the concentration of the solute extrapolated to zero concentration. ItV may be calculated from the measured inherent viscosity.

As used herein, the term “melting point temperature” or “Tm” is the peak minimum of the endotherm on a DSC thermal curve.

As used herein, the term “PET Recycle Standard” refers to the virgin resin used to test the compatibility of a given polyester with PET recycle streams and is defined further herein.

As used herein, the term “Recycle Sample Prep Protocol” refers to the process for making a sample which includes a given polyester and a control PET resin and is defined further herein. A control PET resin may be a PET Recycle Standard resin.

In one embodiment of the invention, the diacid residue is a terephthalic acid monomer. In another embodiment, the terephthalic acid monomer is at least one selected from the group consisting of terephthalic acid and dimethyl terephthalate. Other dicarboxylic acids selected from aliphatic dicarboxylic acids having 3 to 12 carbon atoms, cycloaliphatic dicarboxylic acids having 8 to 14 carbon atoms and aromatic dicarboxylic acids having 8 to 16 carbon atoms may be present in small amounts, although not preferred with typical examples including 1,4-cyclohexane dicarboxylic, phthalic, isophthalic, and 2,6-naphthalene dicarboxylic. The term “terephthalate monomer” is meant to include other corresponding esters, such as phenyl, ethyl, propyl, and butyl, and acid anhydrides although all of these are less preferred.

In certain embodiments, terephthalic acid or an ester thereof, such as, for example, dimethyl terephthalate or a mixture of terephthalic acid residues and an ester thereof can make up a portion or all of the dicarboxylic acid component used to form the polyesters useful in the invention. In certain embodiments, terephthalic acid residues can make up a portion or all of the dicarboxylic acid component used to form the polyesters useful in the invention. In certain embodiments, higher amounts of terephthalic acid can be used in order to produce a higher impact strength polyester. For purposes of this disclosure, the terms “terephthalic acid” and “dimethyl terephthalate” are used interchangeably herein. In one embodiment, dimethyl terephthalate is part or all of the dicarboxylic acid component used to make the polyesters useful in the present invention. In embodiments, ranges of from 70 to 100 mole %; or 80 to 100 mole %; or 90 to 100 mole %; or 99 to 100 mole %; or 100 mole % terephthalic acid and/or dimethyl terephthalate and/or mixtures thereof may be used.

In addition to terephthalic acid, the dicarboxylic acid component of the polyesters useful in the invention can comprise up to 10 mole %, up to 5 mole %, or up to 1 mole % of one or more modifying aromatic dicarboxylic acids. Yet another embodiment contains 0 mole % modifying aromatic dicarboxylic acids. Thus, if present, it is contemplated that the amount of one or more modifying aromatic dicarboxylic acids can range from any of these preceding endpoint values including, for example, 0.01 to 10 mole %, from 0.01 to 5 mole % and from 0.01 to 1 mole %. In one embodiment, modifying aromatic dicarboxylic acids that may be used in the present invention include but are not limited to those having up to 20 carbon atoms, and which can be linear, para-oriented, or symmetrical. Examples of modifying aromatic dicarboxylic acids which may be used in this invention include, but are not limited to, isophthalic acid, 4,4′-biphenyldicarboxylic acid, 1,4-, 1,5-, 2,6-, 2,7-naphthalenedicarboxylic acid, and trans-4,4′-stilbenedicarboxylic acid, and esters thereof. In one embodiment, the modifying aromatic dicarboxylic acid is isophthalic acid.

The carboxylic acid component of the polyesters useful in the invention can be further modified with up to 10 mole %, such as up to 5 mole % or up to 1 mole % of one or more aliphatic dicarboxylic acids containing 2-16 carbon atoms, such as, for example, cyclohexanedicarboxylic, malonic, succinic, glutaric, adipic, pimelic, suberic, azelaic and dodecanedioic dicarboxylic acids. Certain embodiments can also comprise 0.01 to 10 mole %, such as 0.1 to 10 mole %, 1 or 10 mole %, 5 to 10 mole % of one or more modifying aliphatic dicarboxylic acids. Yet another embodiment contains 0 mole % modifying aliphatic dicarboxylic acids. The total mole % of the dicarboxylic acid component is 100 mole %. In one embodiment, adipic acid and/or glutaric acid are provided in the modifying aliphatic dicarboxylic acid component of the invention.

Esters of terephthalic acid and the other modifying dicarboxylic acids or their corresponding esters and/or salts may be used instead of the dicarboxylic acids. Suitable examples of dicarboxylic acid esters include, but are not limited to, the dimethyl, diethyl, dipropyl, diisopropyl, dibutyl, and diphenyl esters. In one embodiment, the esters are chosen from at least one of the following: methyl, ethyl, propyl, isopropyl, and phenyl esters.

In one embodiment of the invention, the diol component of the inventive copolyester is comprised of ethylene glycol residues (EG), diethylene glycol residues (DEG), and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD). In another embodiment, the diol component of the copolyester is comprised of ethylene glycol residues, diethylene glycol residues, and 1,4-cyclohexanedimethanol residues.

Additional aliphatic, alicyclic, and aralkyl glycols may be present in small amounts with examples including 1,2-propandiol also known in the trade as propylene glycol, 1,3-propanediol, 1,4-butanediol, 1,5-pentanediol, 1,6-hexanediol, 2,2-dimethyl-1,3-propanediol, 1,2-cyclohexane dimethanol, 1,3-cyclohexane dimethanol, 2,2,4,4-tetramethyl-1,3-cyclobutanediol, and p-xylylenediol. Diols containing a plurality of ether linkages, such as triethylene glycol and tetraethylene glycol are acceptable in small amounts.

In other embodiments of the invention, the amount of ethylene glycol residues in the copolyester can range from about 85 to about 95 mol %, about 85 to about 94 mol %, about 85 to about 93 mol %, about 85 to about 92 mol %, about 85 to about 91 mol %, about 85 to about 90 mol %, about 85 to 89 mol %, about 86 to about 96 mol %, about 86 to about 95 mol %, about 86 to about 94 mol %, about 86 to about 93 mol %, about 86 to about 92 mol %, about 86 to about 91 mol %, about 86 to about 90 mol %, about 87 to about 96 mol %, about 87 to about 95 mol %, about 87 to about 94 mol %, about 87 to about 93 mol %, about 87 to about 92 mol %, about 87 to about 91 mol %, about 88 to about 96 mol %, about 88 to about 95 mol %, about 88 to about 94 mol %, about 88 to about 93 mol %, about 88 to about 92 mol %, about 89 to about 96 mol %, about 89 to about 95 mol %, about 89 to about 94 mol %, about 89 to about 93 mol %, about 90 to about 96 mol %, about 90 to about 95 mol %, about 90 to about 94 mol %, about 91 to about 96 mol %, and about 91 to about 95 mol %.

In other embodiments of the invention, the inventive copolyester can comprise about 4 to about 14 mole %, about 4 to about 13 mole %, about 4 to about 12 mole %, about 4 to about 11 mole %, about 4 to about 10 mole %, about 4 to about 9 mole %, about 4 to about 8 mole %, about 5 to about 15 mol %, about 5 to about 14 mole %, about 5 to about 13 mole %, about 5 to about 12 mole %, about 5 to about 11 mole %, about 5 to about 10 mole %, about 5 to about 9 mole %, about 6 to about 15 mole %, about 6 to about 14 mole %, about 6 to about 13 mole %, about 6 to about 12 mole %, about 6 to about 11 mole %, about 6 to about 10 mole %, about 7 to about 15 mole %, about 7 to about 14 mole %, about 7 to about 13 mole %, about 7 to about 12 mole %, about 7 to about 11 mole %, about 8 to about 15 mole %, about 8 to about 14 mole %, about 8 to about 13 mole %, about 8 to about 12 mole %, about 9 to about 15 mole %, about 9 to about 14 mole %, about 9 to about 13 mole %, about 9 to about 12 mole %, about 10 to about 15 mole %, and about 10 to about 14 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD).

In other embodiments of the invention, the inventive copolyester can comprise about 4 to about 14 mole %, about 4 to about 13 mole %, about 4 to about 12 mole %, about 4 to about 11 mole %, about 4 to about 10 mole %, about 4 to about 9 mole %, about 4 to about 8 mole %, about 5 to about 15 mol %, about 5 to about 14 mole %, about 5 to about 13 mole %, about 5 to about 12 mole %, about 5 to about 11 mole %, about 5 to about 10 mole %, about 5 to about 9 mole %, about 6 to about 15 mole %, about 6 to about 14 mole %, about 6 to about 13 mole %, about 6 to about 12 mole %, about 6 to about 11 mole %, about 6 to about 10 mole %, about 7 to about 15 mole %, about 7 to about 14 mole %, about 7 to about 13 mole %, about 7 to about 12 mole %, about 7 to about 11 mole %, about 8 to about 15 mole %, about 8 to about 14 mole %, about 8 to about 13 mole %, about 8 to about 12 mole %, about 9 to about 15 mole %, about 9 to about 14 mole %, about 9 to about 13 mole %, about 9 to about 12 mole %, about 10 to about 15 mole %, and about 10 to about 14 mole % of a combination of diethylene glycol (DEG) residues and 1,4-cyclohexanedimethanol residues (CHDM).

In other embodiment of the invention, the amount of germanium present in the copolyester is at a concentration of about 5 to about 450 ppm, about 5 to about 400 ppm, about 5 to about 350 ppm, about 5 to about 300 ppm, about 5 to about 250 ppm, about 5 to about 200 ppm, about 5 to about 150 ppm, about 5 to about 150 ppm, 10 to about 450 ppm, about 10 to about 400 ppm, about 10 to about 350 ppm, about 10 to about 300 ppm, about 10 to about 250 ppm, about 10 to about 200 ppm, about 10 to about 150 ppm, about 10 to about 100 ppm, about 25 to about 450 ppm, about 25 to about 400 ppm, about 25 to about 350 ppm, about 25 to about 300 ppm, about 25 to about 250 ppm, about 25 to about 200 ppm, about 25 to about 150 ppm, about 25 to about 100 ppm, 50 to about 450 ppm, about 50 to about 400 ppm, about 50 to about 350 ppm, about 50 to about 300 ppm, about 50 to about 250 ppm, about 50 to about 200 ppm, about 50 to about 150 ppm, or about 50 to about 100 ppm.

When 1,4-cyclohexanedimethanol is employed as part of the glycol component, the 1,4-cyclohexanedimethanol may be cis, trans, or a mixture thereof. The molar ratio of cis/trans 1,4-cyclohexanedimethanol can vary within the range of 50/50 to 0/100 or 40/60 to 20/80. In one embodiment, the 1,4-cyclohexanedimethanol has a cis/trans ratio of 60:40 to 40:60 or a cis/trans ratio of 70:30 to 30:70. In another embodiment, the trans-cyclohexanedimethanol can be present in an amount of 60 to 80 mole % and the cis-cyclohexanedimethanol can be present in an amount of 20 to 40 mole % wherein the total percentages of cis-cyclohexanedimethanol and trans-cyclohexanedimethanol is equal to 100 mole %. In particular embodiments, the trans-cyclohexanedimethanol can be present in an amount of 60 mole % and the cis-cyclohexanedimethanol can be present in an amount of 40 mole %. In particular embodiments, the trans-cyclohexanedimethanol can be present in an amount of 70 mole % and the cis-cyclohexanedimethanol can be present in an amount of 30 mole %.

In one embodiment, the glycol component of the polyester portion of the polyester compositions useful in the invention can contain up to 10 mole %, or 9 mole %, or 8 mole %, or 7 mole %, or 6 mole %, or less of one or more modifying glycols which are not 2,2,4,4-tetramethyl-1,3-cyclobutanediol, ethylene glycol, diethylene glycol, 1,4-cyclohexanedimethanol, or monopropylene glycol. In one embodiment, the glycol component of the polyester portion of the polyester compositions useful in the invention can contain up to 5 mole %, or 4 mole %, or 3 mole %, or 2 mole %, or 1 mole %, or less of one or more modifying glycols which are not 2,2,4,4-tetramethyl-1,3-cyclobutanediol, ethylene glycol, diethylene glycol, 1,4-cyclohexanedimethanol, or monopropylene glycol. In certain embodiments, the polyesters useful in the invention can contain 3 mole % or less of one or more modifying glycols. In another embodiment, the polyesters useful in the invention can contain 2 mole % or less of one or more modifying glycols. In another embodiment, the polyesters useful in the invention can contain 0 mole % modifying glycols. It is contemplated however that some other glycol residuals may form in situ. For example, a certain amount of DEG will typically be formed in situ during the polymerization reactions.

The formation of DEG from EG is a side reaction that occurs during the melt phase synthesis of polyesters. Most often DEG is undesirable for having a negative impact on properties, such as weathering and toughness.

Germanium also tends to increase the formation of DEG and a surprising aspect of this invention is that it lowers the melting point effectively like CHDM, but does not excessively decrease the crystallization halftime to prevent the molding of thick wall containers. For this reason, it is not necessary to minimize the formation of DEG to a level less than 2 mole % as disclosed in US 2013/0029068 A1.

In embodiments, modifying glycols for use in the polyesters, if used, can include diols other than 2,2,4,4-tetramethyl-1,3-cyclobutanediol, ethylene glycol, diethylene glycol, 1,4-cyclohexanedimethanol, or monopropylene glycol and can contain 2 to 16 carbon atoms. Examples of modifying glycols include, but are not limited to, 1,2-propanediol, 1,3-propanediol, neopentyl glycol, isosorbide, 1,4-butanediol, 1,5-pentanediol, 1,6-hexanediol, p-xylene glycol, polytetramethylene glycol, and mixtures thereof. In another embodiment, the modifying glycols include, but are not limited to, at least one of 1,3-propanediol and 1,4-butanediol.

In some embodiments, the copolyesters according to the invention can comprise from 0 to 10 mole percent, for example, from 0.01 to 5 mole percent, from 0.01 to 1 mole percent, from 0.05 to 5 mole percent, from 0.05 to 1 mole percent, or from 0.1 to 0.7 mole percent, based the total mole percentages of either the diol or diacid residues; respectively, of one or more residues of a branching monomer, also referred to herein as a branching agent, having 3 or more carboxyl substituents, hydroxyl substituents, or a combination thereof. In certain embodiments, the branching monomer or agent may be added prior to and/or during and/or after the polymerization of the polyester. In embodiments, the polyester(s) useful in the invention can thus be linear or branched.

Examples of branching monomers include, but are not limited to, multifunctional acids or multifunctional alcohols such as trimellitic acid, trimellitic anhydride, pyromellitic dianhydride, trimethylolpropane, glycerol, pentaerythritol, citric acid, tartaric acid, 3-hydroxyglutaric acid and the like. In one embodiment, the branching monomer residues can comprise 0.1 to 0.7 mole percent of one or more residues chosen from at least one of the following: trimellitic anhydride, pyromellitic dianhydride, glycerol, sorbitol, 1,2,6-hexanetriol, pentaerythritol, trimethylolethane, and/or trimesic acid. The branching monomer may be added to the polyester reaction mixture or blended with the polyester in the form of a concentrate as described, for example, in U.S. Pat. Nos. 5,654,347 and 5,696,176, whose disclosure regarding branching monomers is incorporated herein by reference.

The copolyesters of the invention can comprise at least one chain extender. Suitable chain extenders include, but are not limited to, multifunctional (including, but not limited to, bifunctional) isocyanates, multifunctional epoxides, including for example epoxylated novolacs, and phenoxy resins. In certain embodiments, chain extenders may be added at the end of the polymerization process or after the polymerization process. If added after the polymerization process, chain extenders can be incorporated by compounding or by addition during conversion processes such as injection molding or extrusion. The amount of chain extender used can vary depending on the specific monomer composition used and the physical properties desired but is generally about 0.1 percent by weight to about 10 percent by weight, such as about 0.1 to about 5 percent by weight, based on the total weight of the copolyester.

Multifunctional reactants with at least three functional groups will result in branching of the copolyester and may optionally be present in minor amounts to facilitate molecular weight build up kinetics for the practice of this invention. Higher levels above 1.0 mole % are less preferred as they lead to increased brittleness. Suitable multifunctional reactants include trimellitic acid, trimellitic anhydride, pyromellitic acid, pyromellitic dianhydride, pentaerythritol, glycerol, trimethylpropane (TMP), trimethylolethane (TME), erythritol, threitol, dipentaerythritol, sorbitol, and dimethylolpropionic acid.

### Copolyester Useful in Recycle PET

The inventive composition can be readily included in the overall PET recycle stream. As actual recycle streams of PET may have variability, testing for compatibility with the overall PET recycle stream is done using a virgin PET Recycle Standard resin. The PET Recycle Standard resin is defined herein as a PET resin comprising 96 to 99.5 mole percent terephthalic acid residues and 0.5 to 4.0 mole percent isophthalic acid residues and 100 mole percent ethylene glycol residues based upon 100 mole percent acid residues and 100 mole percent glycol residues (one skilled in the art recognizes that these PET polyesters contain a small amount of DEG produced in situ or added to maintain a constant minimal amount of DEG; the DEG is counted as part of the 100 mole percent of EG). The Association of Postconsumer Plastic Recyclers has developed the PET Critical Guidance Document (“CGD”) for evaluating the compatibility of innovation polyesters with the PET recycle stream. The PET Recycle Standard resin defined above includes, but is not limited to, the named PET Control Resins listed in the CGD and reproduced in the table below.

| For Low IV, water bottle | For CSD and non-water bottle |
|---|---|
| innovation, named PET | innovations, named PET |
| Control Resins: | Control Resins: |
| Eastman aqua PET RH314 | Eastman PET CB12 |
| Invista Polyclear Splash, 3301 | Invista Polyclear Refresh, 1101 |
| M&G Cleartuf Turbo II | M&G Cleartuf MAX |
| Wellman HP 807 | Wellman HP 806 |
| OAK Lasar + W L44A | OAK Laser + B95A |

The CGD includes a procedure for preparing samples of blends of an innovation resin and one of several named PET Control resins to is in various tests. The Recycle Sample Prep Protocol is based upon, but not limited to, the CGD procedure. The Recycle Sample Prep Protocol is the procedure by which a polyester and a Standard PET Recycle resin are combined and processed before measuring the melting point temperature. The Recycle Sample Prep Protocol is defined as the following Steps 1) through 5).

1) The test polyester and a control PET resin are independently dried, extruded, re-pelletized, and crystallized. Extrusion processing is conducted according to typical PET processing conditions (240-280° C. barrel temperature settings). Crystallization is conducted at approximately 160° C.

2) A pellet-pellet blend is formed by combining the re-pelletized test polyester and the re-pelletized control PET resin from Step 1. The blend is dried at 160° C. for at least 4 hours.

3) The dry blend from Step 2 is extruded, re-pelletized, and crystallized. Extrusion processing is conducted according to typical PET processing conditions (240 to 280° C. barrel temperature settings). Crystallization is conducted at approximately 160° C.

4) The crystallized blend from Step 3 is solid-stated (195-215° C.) until a nominal 0.80 intrinsic viscosity (ItV) is obtained as measured by ASTM D 4603.

5) DSC melting point temperature measurement is conducted on the solid-stated blend from Step 4, according to Method 2 (at 10° C./min, on the second heat scan following annealing for 2 minutes at 280° C.).

Note that the when the control PET resin is one of the named PET Control Resins listed in the CGD and blended with an innovative resin (test polyester) at a level of 0 weight percent, 25 weight percent, or 50 weight percent of the innovative resin, the melting point temperature (Tm) of the blend follows the CGD test, 3.1 Melting Point Test, which lists a critical value of 235° C. to 255° C. for the melting point temperature. The control PET resin can be the PET Recycle Standard resin as defined herein above, and the test polyesters can be copolyesters of this invention.

In one embodiment the melting point temperature Tm of a blend comprising 50 weight percent of the copolyesters of this invention with 50 weight percent of a PET Recycle Standard resin and prepared according to the above

Recycle Sample Prep Protocol is in the range of 200 to 270° C.; 200 to 260° C.; 200 to 255° C.; 200 to 250° C.; 200 to 245° C.; 200 to 240° C.; 200 to 235° C.; 210 to 270° C.; 210 to 260° C.; 210 to 255° C.; 210 to 250° C.; 210 to 245° C.; 210 to 240° C.; 210 to 235° C.; 220 to 270° C.; 220 to 260° C.; 220 to 255° C.; 220 to 250° C.; 220 to 245° C.; 220 to 240° C.; 220 to 235° C.; 225 to 270° C.; 225 to 260° C.; 225 to 255° C.; 225 to 250° C.; 225 to 245° C.; 225 to 240° C.; 225 to 235° C.; 230 to 270° C.; 230 to 260° C.; 230 to 255° C.; 230 to 250° C.; 230 to 245° C.; 230 to 240° C.; 230 to 235° C.; 235 to 270° C.; 235 to 260° C.; 235 to 255° C.; 235 to 250° C.; 235 to 245° C.; or 235 to 240° C.

It is contemplated that compositions useful in the invention can possess at least one of the inherent viscosity ranges described herein and at least one of the monomer ranges for the compositions described herein unless otherwise stated. It is also contemplated that the copolyesters of this invention when blended with the PET Recycle Standard can have at least one of the melting point temperature, Tm, ranges described herein and at least one of the monomer ranges for the composition described herein unless otherwise stated. It is contemplated that the copolyesters of this invention can possess at least one of the monomer ranges for the compositions described herein, and at least one of the inherent viscosity ranges described herein, and, when blended with the PET Recycle Standard, the blend can have at least one of the melting point temperature, Tm, ranges described herein unless otherwise stated.

### Process of Making the Copolyester

The process for preparing polyesters by reacting the dicarboxylic acids and diols typically involves two distinct stages, a combined esterification and transesterification stage followed by a polycondensation stage. The diols, depending on their reactivities and specific process conditions employed, are typically used in molar excesses of 1.01 to 4 moles, preferably 1.01 to 2 moles, per total moles of terephthalate monomers. Less volatile glycols, specifically, 1,4-CHDM and DEG, are advantageously added near stoichiometric balance to achieve target composition, while ethylene glycol is a more volatile glycol that is more readily vaporized, particularly during the vacuum stage and typically added in stoichiometric excess.

In one embodiment, the esterification and/or transesterification reactions are advantageously conducted under an inert atmosphere (e.g., N₂) at a temperature of 150 to 270° C. for 0.5 to 8 hours at atmospheric or greater pressure. Other process conditions include conducting the esterification and transesterification under an inert atmosphere at a temperature of from 200 to 260° C. for 1 to 4 hours at atmospheric or greater pressure. The esterification and transesterification can be conducted in the presence of any catalyst known in the art or without catalyst.

In the second stage of the process, polycondensation, is conducted under reduced pressure of 0.1 to 100 torr at a temperature of 220 to 310° C. in the presence of a germanium catalyst. In other embodiments, the temperature during the polycondensation ranges from 240 to 290° C. or 260 to 280° C. The duration of the polycondensation can range from 0.1 to 6 hours, 0.5 to 5 hours, 1 to 5 hours, 2 to 5 hours, 3 to 5 hours, or 4 to 5 hours.

Stirring or appropriate conditions are used in both stages to ensure adequate heat transfer, mass transport, and surface renewal of the reaction mixture. The reactions for both stages are facilitated by one or more appropriate catalysts. This invention can use known ester exchange catalysts to react the terephthalate monomer with the glycols, including metal acetates, such as manganese acetate, zinc acetate, aluminum acetate, cobalt acetate and so forth. Manganese is preferred. Terephthalic acid is autocatalytic and does not require a catalyst for esterification. In an embodiment of the invention, titanium and tin are known catalysts that may be not suitable for the practice of this invention as they lead to higher color although they could be present in small detectable amounts.

Germanium is used as the polycondensation catalyst in any soluble form known in the art. For example, germanium catalyst can include, but not limited to, oxide, alkoxy, alkyl and halo germanates. Germanium catalysts are disclosed in U.S. Pat. Nos. 2,578,660; 3,074,913; 3,377,320; 3,346,541; 3,459,711; 3,497,474; 3,497,475; 3,511,811; 3,651,017; 3,647,362; and 3,842,043, herein incorporated by reference to the extent these patents do not contradict this specification.

Suitable germanium compounds include, for example, germanium (IV) oxide, amorphous or crystal germanium dioxide (hexagonal and tetragonal), germanium glycoxide, such as germanium ethylene glycoxide, germanium alkoxide and its derivatives, such as germanium ethoxide, germanium isopropoxide, germanium carboxylate, such as the acetate, germanium tetrahalide such as the tetrachloride and other known germanium compounds being readily and uniformly soluble in ethylene glycol or in the reaction mixture. In one embodiment, the germanium catalyst is hexagonal amorphous or crystal germanium dioxide since it yields copolyesters having less haze.

These compounds can be employed in the form known in the art, for example, amorphous germanium dioxide; a solid such as finely powdered crystal germanium dioxide having an average-particle size of no more than 3; an aqueous solution; an ethylene glycol solution; an aqueous germanium solution; or by directly dissolving germanium compounds in ethylene glycol in the presence of alkali metal salt or alkaline earth metal salt.

The amount of germanium catalyst added in the polymerization can range from 25 to 1000 ppm based on the yield of final copolyester. In other embodiments, the amount of germanium catalyst can range from 50 to 950 ppm, 50 to 900 ppm, 50 to 850 ppm, 50 to 800 ppm, 50 to 750 ppm, 50 to 700 ppm, 50 to 650 ppm, 50 to 600 ppm, 50 to 550 ppm 50 to 500 ppm, 50 to 450 ppm, 100 to 950 ppm, 100 to 900 ppm, 100 to 850 ppm, 100 to 800 ppm, 100 to 750 ppm, 100 to 700 ppm, 100 to 650 ppm, 100 to 600 ppm, 100 to 550 ppm 100 to 500 ppm, 100 to 450 ppm, 150 to 950 ppm, 150 to 900 ppm, 150 to 850 ppm, 150 to 800 ppm, 150 to 750 ppm, 150 to 700 ppm, 150 to 650 ppm, 150 to 600 ppm, 150 to 550 ppm, 150 to 500 ppm, 150 to 450 ppm, 200 to 950 ppm, 200 to 900 ppm, 200 to 850 ppm, 200 to 800 ppm, 200 to 750 ppm, 200 to 700 ppm, 200 to 650 ppm, 200 to 600 ppm, 200 to 550 ppm, 200 to 500 ppm, 200 to 450 ppm, 250 to 950 ppm, 250 to 900 ppm, 250 to 850 ppm, 250 to 800 ppm, 250 to 750 ppm, 250 to 700 ppm, 250 to 650 ppm, 250 to 600 ppm, 250 to 550 ppm, 250 to 500 ppm, 250 to 450 ppm, 300 to 950 ppm, 300 to 900 ppm, 300 to 850 ppm, 300 to 800 ppm, 300 to 750 ppm, 300 to 700 ppm, 300 to 650 ppm, 300 to 600 ppm, 300 to 550 ppm, 300 to 500 ppm, and 300 to 450 ppm.

There are additional process variations that are in scope based on what is known for polyesters. For example, staged addition or pre-reaction of glycols is within scope. Also, it is acceptable to add 1,4-CHDM to post-consumer or post-industrial PET to obtain a copolyester of terephthalate, EG, and CHDM.

A novel aspect of this invention is that germanium catalyst although typically added in high amounts compared to titanium/antimony does not tend to decrease the crystallization half time. This is unexpected since titanium and antimony were at lower concentrations for the comparative examples.

It will be apparent to persons skilled in the art that copolyesters of the present invention can be prepared using recycled monomers that have been recovered by depolymerization of scrap or post-consumer polyesters, or a combination of virgin and recycled monomers. Processes for the depolymerization of polyesters into their component monomers are well-known. For example, one known technique is to subject the polyester, typically PET, to methanolysis in which the polyester is reacted with methanol to produce dimethyl terephthalate (“DMT”), dimethyl isophthalate, ethylene glycol (“EG”), and 1,4-cyclohexanedimethanol (“CHDM”), depending on the composition of the polyester. Some representative examples of the methanolysis of PET are described in U.S. Pat. Nos. 3,321,510; 3,776,945; 5,051,528; 5,298,530; 5,576,456; and 6,262,294, which are incorporated herein by reference. In a typical methanolysis process, the scrap PET resin is dissolved in oligomers of dimethyl terephthalate and ethylene glycol. Superheated methanol is then passed through the solution and reacts with the dissolved polyester and polyester oligomers to form dimethyl terephthalate and ethylene glycol. These monomers can be recovered by distillation, crystallization, or a combination thereof. For example, U.S. Pat. No. 5,498,749 describes the recovery and purification of dimethyl terephthalate from depolymerization process mixtures containing 1,4-cyclohexanedimethanol.

Glycolysis is another commonly used method of depolymerizing polyesters. A typical glycolysis process can be illustrated with particular reference to the glycolysis of PET, in which waste PET is dissolved in and reacted with a glycol, typically ethylene glycol, to form a mixture of dihydroxyethyl terephthalate and low molecular weight terephthalate oligomers. This mixture is then subjected to a transesterification with a lower alcohol, i.e., methanol to form dimethyl terephthalate and ethylene glycol. The DMT and ethylene glycol can be recovered and purified by distillation or a combination of crystallization and distillation. Some representative examples of glycolysis methods can be found in U.S. Pat. Nos. 3,907,868; 6,706,843; and 7,462,649, which are incorporated herein by reference.

The recycled DMT and ethylene glycol may be used directly in polycondensation reactions to prepare polyesters and copolyesters. The DMT can be hydrolyzed to prepare terephthalic acid or hydrogenated to CHDM using known procedures. The TPA and CHDM may then be repolymerized into copolyesters.

The recycled monomers can be repolymerized into polyesters using typical polycondensation reaction conditions well-known to persons skilled in the art. They may be made by continuous, semi-continuous, and batch modes of operation and may utilize a variety of reactor types.

Examples of suitable reactor types include, but are not limited to, stirred tank, continuous stirred tank, slurry, tubular, wiped-film, falling film, or extrusion reactors. The polyesters may comprise only recycled monomers or a mixture of recycled and virgin monomers. For example, the proportion of the diacid and diol residues that are from recycled monomers can each range from about 0.5 to about 100 mole percent, based on a total of 100 mole percent diacid residues and 100 mole percent diol residues. When prepared from recycled monomers of sufficient purity, the copolyesters of this invention are indistinguishable from the same copolyesters prepared from virgin monomers.

The copolyesters of this invention can have a crystallization half time of greater than 1 minute, greater than 2 minutes, greater than 3 minutes, greater than 4 minutes, or greater than 5 minutes at 140° C. as measured by the method described in the Examples. In other embodiments, the copolyesters of this invention can have a crystallization half time of greater than 1 minute, greater than 2 minutes, greater than 3 minutes, greater than 4 minutes, or greater than 5 minutes at 160° C. In other embodiments, the copolyesters of this invention can have a crystallization half time of greater than 1 minute, greater than 2 minutes, greater than 3 minutes, greater than 4 minutes, or greater than 5 minutes at 180° C. as measured by the method described in the Examples. In yet other embodiments, the copolyesters of this invention have a crystallization half time of greater than 1 minute, greater than 2 minutes, greater than 3 minutes, greater than 4 minutes, or greater than 5 minutes at 140° C., 160° C., and 180° C. as measured by the method described in the Examples. These crystallization half times allow the copolyesters to be utilized in thick-walled containers of various types. In one embodiment of the invention, cosmetics containers comprise the copolyesters of this invention.

In embodiments of the invention, certain agents which colorize the polymer can be added to the melt. In one embodiment, a bluing toner is added to the melt in order to reduce the b* of the resulting polyester polymer melt phase product. Such bluing agents include blue inorganic and organic toner(s), In addition, red toner(s) can also be used to adjust the a* color. Organic toner(s), e.g., blue and red organic toner(s), such as those toner(s) described in U.S. Pat. Nos. 5,372,864 and 5,384,377, which are incorporated by reference in their entirety, can be used. The organic toner(s) can be fed as a premix composition. The premix composition may be a neat blend of the red and blue compounds or the composition may be pre-dissolved or slurried in one of the polyester's raw materials, e.g., ethylene glycol.

The total amount of toner components added can depend on the amount of inherent yellow color in the base polyester and the efficacy of the toner. In one embodiment, a concentration of up to about 15 ppm of combined organic toner components and a minimum concentration of about 0.5 ppm are used. In one embodiment, the total amount of bluing additive can range from 0.5 to 10 ppm. In an embodiment, the toner(s) can be added to the esterification zone or to the polycondensation zone. Preferably, the toner(s) are added to the esterification zone or to the early stages of the polycondensation zone, such as to a prepolymerization reactor.

The invention further relates to a polymer blend. In embodiments, the blend comprises:
(a) from 5 to 95 weight % of at least one of the copolyesters described above; and
(b) from 5 to 95 weight % of at least one polymeric component.

Suitable examples of the polymeric components include, but are not limited to, nylon; polyesters different than those described herein such as PET; polyamides such as ZYTEL® from DuPont; polystyrene; polystyrene copolymers; styrene acrylonitrile copolymers; acrylonitrile butadiene styrene copolymers; poly(methylmethacrylate); acrylic copolymers; poly(ether-imides) such as ULTEM® (a poly(ether-imide) from General Electric); polyphenylene oxides such as poly(2,6-dimethylphenylene oxide) or poly(phenylene oxide)/polystyrene blends such as NORYL 100 (a blend of poly(2,6-dimethylphenylene oxide) and polystyrene resins from General Electric); polyphenylene sulfides; polyphenylene sulfide/sulfones; poly(ester-carbonates); polycarbonates such as LEXAN® (a polycarbonate from General Electric); polysulfones; polysulfone ethers; and poly(ether-ketones) of aromatic dihydroxy compounds; or mixtures of any of the foregoing polymers. The blends can be prepared by conventional processing techniques known in the art, such as melt blending or solution blending.

In embodiments, the copolyester and the polymer blend compositions can also contain from 0.01 to 25% by weight of the overall composition common additives such as colorants, toner(s), dyes, mold release agents, flame retardants, plasticizers, nucleating agents, stabilizers, including but not limited to, UV stabilizers, thermal stabilizers other than the phosphorus compounds describe herein, and/or reaction products thereof, fillers, and impact modifiers. Examples of commercially available impact modifiers include, but are not limited to, ethylene/propylene terpolymers, functionalized polyolefins such as those containing methyl acrylate and/or glycidyl methacrylate, styrene-based block copolymeric impact modifiers, and various acrylic core/shell type impact modifiers. Residues of such additives are also contemplated as part of the polyester composition.

Reinforcing materials may be added to the compositions of this invention. The reinforcing materials may include, but are not limited to, carbon filaments, silicates, mica, clay, talc, titanium dioxide, Wollastonite, glass flakes, glass beads and fibers, and polymeric fibers and combinations thereof. In one embodiment, the reinforcing materials include glass, such as, fibrous glass filaments, mixtures of glass and talc, glass and mica, and glass and polymeric fibers.

In one aspect, the invention relates to the film(s) and/or sheet(s) comprising the polyester compositions and/or polymer blends of the invention. The methods of forming the polyesters and/or blends into film(s) and/or sheet(s) are well known in the art. Examples of film(s) and/or sheet(s) of the invention including but not limited to extruded film(s) and/or sheet(s), calendered film(s) and/or sheet(s), compression molded film(s) and/or sheet(s), solution casted film(s) and/or sheet(s). Methods of making film and/or sheet include but are not limited to extrusion, calendering, compression molding, and solution casting.

When polyesters according to embodiments of the present invention are extrusion blow molded at one or more of the high shear rates discussed above, they surprisingly exhibit little or no haze. In particular, extrusion blow molded articles made from the inventive polyesters discussed herein at one or more of the shear rates discussed above can exhibit sidewall haze values of less than 15%, less than 10%, less than 7%, less than 5%, or less than 4%. Haze is measured on sidewalls of molded articles according to ASTM D 1003, Method A, and is calculated as a percentage, from the ratio of diffuse transmittance to total light transmittance. A BYK-Gardner HazeGuard Plus is used to measure haze.

In one embodiment, the extrusion blow molded article is formed entirely of the copolyester of this invention. In other embodiments, the copolyester of this invention can be mixed with another composition prior to extrusion blow molding. However, even when the copolyester of this invention is mixed with another composition prior to extrusion blow molding, the resulting extrusion blow molded articles can still contain the novel copolyester in an amount of at least 90 weight %, at least 95 weight %, at least 98 weight %, or at least 99 weight %.

In one embodiment, the copolyesters of this invention degradation in lhV during extrusion blow molding (i.e., the IhV of the polyester before the EBM process minus the IhV of the article) is less than 0.1 dl/g, less than 0.075 dl/g, less than 0.05 dl/g, less than 0.03 dl/g, less than 0.02 dl/g.

It is contemplated that the compositions, inherent viscosities, and blend melting point temperatures, listed herein above for a polyester useful for the extrusion blow molded article invention, apply also to the process for extrusion blow molding a polyester.

The equipment used to form the extrusion blow molded article is not particularly limiting and includes any equipment known to one skilled in the art for such purpose. The two types of extrusion blow molding that involve a hanging parison are referred to as “shuttle” and “intermittent” processes. In a shuttle process, the mold is situated on a moving platform that moves the mold up to the extruder die, closes it around the parison while cutting off a section, and then moves away from the die to inflate, cool, and eject the bottle. Due to the mechanics of this process, the polymer is continuously extruded through the die at a relatively slow rate. By contrast, the mold in an intermittent process is fixed below the die opening and the full shot weight (the weight of the bottle plus flash) of polymer must be rapidly pushed through the die after the preceding bottle is ejected but before the current bottle is inflated. Intermittent processes can either utilize a reciprocating screw action to push the parison, or the extrudate can be continuously extruded into a cavity which utilizes a plunger to push the parison.

In a very different type of extrusion blow molding process, a 4 to 20 ft diameter wheel moving at 1 to 10 revolutions per minute grabs the parison as it extrudes from the die and lays it in molds attached to the wheel's outer circumference. Mold closure, parison inflation, cooling, and ejection of the bottle occurs sequentially as the wheel turns. In this “wheel process,” the parison is actually pulled from the die by the wheel whereby good melt strength is required to prevent thinning of the parison during both pulling as well as subsequent blowing. The parison in a wheel process can exit the die in either an upward or downward direction and melt strength will be more crucial during upward extrusion due to the effects of gravity. Because of the continuous nature of this “wheel” process, polymer can be extruded from the die at very high speeds.

The copolyesters of this invention can be used to produce any article known in the art. Examples of potential articles made from film and/or sheet useful in the invention include, but are not limited, to thermoformed sheet, graphic arts film, outdoor signs, ballistic glass, skylights, coating(s), coated articles, painted articles, shoe stiffeners, laminates, laminated articles, medical packaging, general packaging, shrink films, pressure sensitive labels, stretched or stretchable films or sheets, uniaxially or biaxially oriented films, and/or multiwall films or sheets.

In one aspect, the invention relates to injection molded articles comprising the polyester compositions and/or polymer blends of the invention. Injection molded articles can include injection stretch blow molded bottles, sun glass frames, lenses, sports bottles, drinkware, food containers, medical devices and connectors, medical housings, electronics housings, cable components, sound dampening articles, cosmetic containers, wearable electronics, toys, promotional goods, appliance parts, automotive interior parts, and consumer houseware articles.

In embodiments of the invention, certain polyesters and/or polyester compositions of the invention can have a unique combination of all of the following properties: certain notched Izod impact strength, certain inherent viscosities, certain glass transition temperature (Tg), certain flexural modulus, good clarity, and good color.

Because of the long crystallization half-times (e.g., greater than 1 minutes) at 170° C. exhibited by the copolyesters of the present invention, it can be possible to produce articles, including but not limited to, injection molded parts, injection blow molded articles, injection stretch blow molded articles, extruded film, calendered film, shrink films, pressure sensitive labels, extruded sheet, extrusion blow molded articles, extrusion stretch blow molded articles, and fibers. A thermoformable sheet is an example of an article of manufacture provided by this invention. The polyesters of the invention can be amorphous or semicrystalline. In one aspect, certain polyesters useful in the invention can have relatively low crystallinity. Certain polyesters useful in the invention can thus have a substantially amorphous morphology, meaning that the polyesters comprise substantially unordered regions of polymer.

### Molecular Weight

Inherent viscosity (IhV) for these polyesters is a useful specification for molecular weight as determined according to the ASTM D2857-70 procedure, in a Wagner Viscometer of Lab Glass, Inc., having a ½ mL capillary bulb, using a polymer concentration about 0.5% by weight in 60/40 by weight of phenol/tetrachloroethane. The procedure is carried out by heating the polymer/solvent system at 120° C. for 15 minutes, cooling the solution to 25° C. and measuring the time of flow at 25° C. The IV is calculated from the equation:

$${η}_{inh} = \frac{ln \frac{{t}_{S}}{{t}_{0}}}{C}$$

where:
η: inherent viscosity at 25° C. at a polymer concentration of 0.5 g/100 mL of solvent;
tₛ: sample flow time;
t₀: solvent-blank flow time;
C: concentration of polymer in grams per 100 mL of solvent (0.5)

The units of the inherent viscosity throughout this application are in the deciliters/gram.

In the following examples, a viscosity was measured in tetrachloroethane/phenol (60/40, weight ratio) at 25° C. and calculated in accordance with the following equation:

$${η}_{inh} = \frac{ln ( {η}_{sp} )}{C}$$

wherein, ηₛₚ is a specific viscosity and C is a concentration. The units of IhV are deciliters/g.

The IhV of the polyester is at least 0.2, preferably 0.4-1.0, and more preferably 0.5-0.8.

### EXAMPLES

### Preparation of Examples

Color plaques (0.125-inch thickness) were molded as thick walled parts to measure the thermal properties directly and color measurements that are more representative than crystalline pellets. Pellets of each copolyester were dried at 137° C. under vacuum for 4-5 hours before molding color chips on a BOY22 injection molding machine. The barrel temperature was 270 C with a mold temperature of 85 F.

All thermal tests for these pellets and molded color plaques were completed at standard DSC scans at 10° C./min for the melt. T_{1/2} was measured at 3 different temperatures, 140° C., 160° C. and 180′C. It is a requirement of this invention that the crystallization half-time is longer than 1 minute to allow fabrication of thick-walled parts.

### Molecular Weight

Inherent viscosity (IhV) for these polyesters is a useful specification for molecular weight as determined according to the ASTM D2857-70 procedure, in a Wagner Viscometer of Lab Glass, Inc., having a ½ mL capillary bulb, using a polymer concentration about 0.5% by weight in 60/40 by weight of phenol/tetrachloroethane. The procedure is carried out by heating the polymer/solvent system at 120° C. for 15 minutes, cooling the solution to 25° C. and measuring the time of flow at 25° C. The IV is calculated from the equation:

$${η}_{inh} = \frac{ln \frac{{t}_{S}}{{t}_{0}}}{C}$$

where:
η: inherent viscosity at 25° C. at a polymer concentration of 0.5 g/100 mL of solvent;
tₛ: sample flow time;
t₀: solvent-blank flow time;
C: concentration of polymer in grams per 100 mL of solvent (0.5)

The units of the inherent viscosity throughout this application are in the deciliters/gram.

In the following examples, a viscosity was measured in tetrachloroethane/phenol (60/40, weight ratio) at 25° C. and calculated in accordance with the following equation:

$${η}_{i n h} = \frac{ln ( {η}_{sp} )}{C}$$

wherein ηₛₚ, is a specific viscosity and C is a concentration. The units of IhV are deciliters/g.

The crystallization halftimes were measured using a differential scanning calorimeter (DSC). In these cases, the samples were ramped (20° C./min) to 285° C. and held isothermally for 2 mins. Next, the polymer was quickly dropped to a setpoint isothermal crystallization temperature (140-180° C.) and held until crystallization was completed, denoted by a full endothermic heat flow curve. Half-time was reported as the time from reaching the crystallization temperature to the time that half of the endothermic crystallization peak was formed.

### Example 1: Synthesis of a Copolyester Using Ti/Sb for Polycondensation with DEG <1.5 Mole % (Comparative)

116.5 g (0.6 mole) of DMT, 71.5 g (1.15 mole) of EG, 8.5 g (0.06 mole) of CHDM were charged to a 500-ml round bottom flask and a Ti solution (3.3 g/L, 0.29 mL), an Sb solution (0.022 g/mL, 1.1 mL), and a Mn solution (2.3 g/L, 3.15 mL) were all added to provide a catalytic level of 8 ppm Ti, 200 ppm Sb, and 60 ppm Mn based on theoretical polymer yield. The reaction vessel was then equipped with a glass polymer head to allow with nitrogen/vacuum inlet, glass sidearm to allow removal of volatile by-products and stainless steel stirrer to allow sufficient mass transfer. The sidearm was attached to a condenser that was connected to a vacuum flask. After set-up of the polymerization, all reactions were performed on computer automated polymer rigs equipped with Camile™ software. The flask was purged 2× with nitrogen before immersion in a metal bath that was pre-heated to 200° C. After the contents were at temperature, the agitator was started and maintained at 200 rpm under a gentle nitrogen sweep. The temperature was increased and the raw materials were melted at 220° C. for 10 minutes and after an additional temperature increase the transesterification reaction between the DMT, CHDM and EG was performed at 245° C. for 148 minutes. Methanol was condensed and collected as transesterification proceeded to completion. At the end of the transesterification, a clear, colorless melt with low viscosity was obtained. A solution containing phosphorous stabilizer was then added to the melt in a quantity to provide 50 ppm of phosphorus (P) to the final polyester. After raising the temperature to 255° C., the nitrogen flow was terminated and replaced with a vacuum that was gradually ramped down to 400 torr over 5 minutes and held for 55 minutes. The reaction was continued with a gradual increase in vacuum (reduced from 400 torr to 200 torr, to 4 and finally 0.5 torr) while raising temperature from 265 to 275° C. over the course of 4 hours to obtain desired molecular weight. After cooling to room temperature, analysis of the polymer yielded an IhV of 0.67. The composition was analyzed to contain 10.5 mole % CHDM and 1.1 mole % DEG for a total glycol modification of 11.6 mole %.

### Example 2: Synthesis of a Copolyester Using Ge for Polycondensation with DEG of 4.5 Mole %

97.1 g (0.5 mol) of DMT, 53.3 g (0.86 mol) of EG, 5.94 g (0.04 mol) of CHDM were charged to a 500-ml round bottom flask fitted with a 24/40 ground glass joint and a Mn solution (0.3 wt %, 1.725 g) was added to provide a catalytic level of 60 ppm Mn based on theoretical polymer yield. The reaction vessel was then equipped with a glass polymer head to allow with nitrogen/vacuum inlet, glass sidearm to allow removal of volatile by-products and a stainless steel stirrer to allow sufficient mass transfer. The sidearm was attached to a condenser that was connected to a vacuum flask. After set-up, the polymerization was controlled using a computer equipped with Camile™ software. The flask was purged 2× with nitrogen before immersion in a metal bath that was pre-heated to 200° C. After the contents were at temperature, the agitator was started and maintained at 200 rpm under a gentle nitrogen sweep. The raw materials were melted at 200° C. for 10 minutes and the transesterification reaction between the DMT, CHDM and EG was performed at 200° C. for 60 minutes and at 215° C. for 75 minutes with liberation of methanol. At the end of the transesterification, a clear, colorless, low viscosity melt was obtained. A solution of phosphate ester was then added to the melt in a quantity to provide a target level of 60 ppm in final polymer followed by a GeO₂ solution (3.6 wt %, 0.96 g) with target level of 300 ppm in the final polymer. The temperature was raised to 250° C. and the nitrogen flow terminated and replaced with a vacuum that was gradually ramped down to 400 torr over 2 minutes and held for 30 minutes. The reaction was further performed at lower vacuum (reduced from 400 torr to 150 torr, to 5 torr and finally 0.5 torr) while raising temperature from 250 to 278° C. over the course of 3 hours to obtain desired viscosity. After cooling to room temperature, analysis of the polymer yielded an IhV of 0.684. The composition was analyzed to contain 8.7 mole % CHDM and 4.6 mole % DEG for a total glycol modification of 13.3 mole %.

### Example 3: Synthesis of a Copolyester Using Ge for Polycondensation with DEG of 2.5 Mole %

87.3 g (0.45 mol) of DMT, 53.12 g (0.85 mol) of EG, 6.22 g (0.043 mol) of CHDM were charged to a 500-ml round bottom flask fitted with a 24/40 ground glass joint and a Mn solution (0.3 wt %, 1.73 ml) was added to provide a catalytic level of 50 ppm Mn based on theoretical polymer yield. The reaction vessel was then equipped with a glass polymer head to allow with nitrogen/vacuum inlet, glass sidearm to allow removal of volatile by-products and stainless steel stirrer to allow sufficient mass transfer. The sidearm was attached to a condenser that was connected to a vacuum flask. After set-up, the polymerization was controlled using a computer equipped with Camile™ software. The flask was purged 2× with nitrogen before immersion in a metal bath that was pre-heated to 200° C. After the contents were at temperature, the agitator was started and maintained at 200 rpm under a gentle nitrogen sweep. The raw materials were melted at 200° C. for 10 minutes and the transesterification reaction between the DMT, CHDM and EG was performed at 200° C. for 60 minutes and at 215° C. for 75 minutes with liberation of methanol until completion. At the end of the transesterification, a clear, colorless, low viscosity melt was obtained. A solution of phosphate ester was then added to the melt in a quantity to provide a target level of 30 ppm in final polymer followed by a GeO₂ solution (3.6 wt %, 0.56 ml) with a target level of 250 ppm in the final polymer. The temperature was raised to 265° C. and the nitrogen flow terminated and replaced with a vacuum that was gradually ramped down to 130 torr over 4 minutes and held for 30 minutes. The reaction was further performed at lower vacuum (reduced from 130 torr to 4 torr, and finally 1 torr) while raising temperature from 265 to 280° C. over the course of 200 minutes to obtain the desired molecular weight. After cooling to room temperature, analysis of the polymer yielded an IhV of 0.67. The composition was analyzed to contain 9.5 mole % CHDM and 2.5 mole % DEG for a total glycol modification of 12.0 mole %.

### Example 4: Synthesis of a Copolyester Using Ge for Polycondensation with DEG of 1.5 Mole %

97.1 g (0.50 mole) of DMT, 46.5 g (0.75 mole) of EG, 7.6 g (0.05 mole) of CHDM were charged to a 500-ml round bottom flask fitted with a 24/40 ground glass joint and a Mn solution (0.0055 g/ml, 1002 μl) was added to provide a catalytic level of 55 ppm Mn based on theoretical polymer yield. The reaction vessel was then equipped with a glass polymer head to allow with nitrogen/vacuum inlet, glass sidearm to allow removal of volatile by-products and stainless steel stirrer to allow sufficient mass transfer. The sidearm was attached to a condenser that was connected to a vacuum flask. After set-up, the polymerization was controlled using a computer equipped with Camile™ software. The flask was purged 2× with nitrogen before immersion in a metal bath that was pre-heated to 210° C. After the contents were at temperature, the agitator was started and maintained at 200 rpm under a gentle nitrogen sweep. The raw materials were melted at 210° C. for 5 minutes and the transesterification reaction between the DMT, CHDM and EG was performed at 210° C. for 90 minutes and at 230° C. for 90 minutes with liberation of methanol until completion. At the end of the transesterification, a clear, colorless, low viscosity melt was obtained. A solution of GeO₂ (0.042 g/ml, 0.89 ml) with a target level of 375 ppm in the final polymer. The temperature was raised to 260° C. and the nitrogen flow terminated and replaced with a vacuum that was gradually ramped down to 150 torr over 5 minutes and held for 20 minutes. The reaction was further performed at lower vacuum (reduced from 150 torr to 3 torr) after raising temperature from 260 to 274° C. for 30 minutes. Finally, the temperature was raised from 274° C. to 280° C. and the vacuum lowered to 0.5 torr and held for 105 minutes to obtain the desired molecular weight. After cooling to room temperature, analysis of the polymer yielded an IhV of 0.69. The composition was analyzed to contain 10.9 mole % CHDM and 1.5 mole % DEG for a total glycol modification of 12.4 mole %.

Examples 1 and 2 show how under similar process conditions germanium catalyst results in higher DEG in comparison to a standard catalyst package for polycondensation using titanium (Ti) and Sb (antimony). The comparison of Example 1 and 2 is further illustrated by the lower amount of excess EG used in Example 2 (germanium catalyst) as a lower level of EG should typically lead to the formation of less DEG. It is practical to use 1 mole % or even less DEG as a limit that can be reached for titanium/antimony catalysts that is known in both the copolyester and PET art. Germanium catalyst tends to produce more DEG under similar process conditions with 4 mole % as a typical value, although it is possible to go lower by changing process conditions as shown in Examples 3 and 4, the level of DEG is not as low compared to titanium/antimony. Thus, a lower limit of 1.5 mole % DEG is a placeholder for this invention.

### Examples 5-9: Copolyesters Varying in CHDM: DEG Ratio Having the Same Melting Point

Copolyesters similar in molecular weight were obtained using the procedures described in Examples 1-4 and the results in Table 1 show that either glycol is suitable to provide a melting point above 225° C. in view of the total modification. However, glass transition has some impact as DEG is known to lower Tg supporting that maintaining the same melting point is not obvious.

TABLE 1
Composition and Melting Point Examples

| | | | Total | | | |
|---|---|---|---|---|---|---|
| | Mole % | Mole % | modification | IhV | T_{g} | Tₘ |
| Example | CHDM | DEG | (mole %) | (dl/g) | (° C.) | (° C.) |
| 5 | 10.2 | 1.9 | 12.1 | 0.61 | 78 | 227 |
| 6 | 6.9 | 6.1 | 13.0 | 0.61 | 75 | 227 |
| 7 | 8.1 | 2.0 | 10.1 | 0.63 | 79 | 233 |
| 8 | 6.4 | 4.3 | 10.7 | 0.58 | 78 | 234 |
| 9 | 4.4 | 5.7 | 10.1 | 0.58 | 76 | 234 |

### Examples 10-13: Germanium Catalyst does not Change the Crystallization Rate of Copolyesters Relative to Titanium Antimony Catalyst at a Total Modification of 11 Mole % and 13 Mole % (Comparative)

Copolyesters similar in molecular weight were obtained using the procedures described in Examples 1-3 and the results are provided in Table 2.

TABLE 2
Comparison of Crystallization Half-times for
Germanium and Ti/Sb Catalyzed Copolyesters

| | | | | Total | | | | |
|---|---|---|---|---|---|---|---|---|
| | | Mole | Mole | modifica- | | T_{1/2} (minutes) | | |
| Exam- | Cata- | % | % | tion | IhV | 140° | 160° | 180° |
| ple | lyst | CHDM | DEG | (mole %) | (dl/g) | C. | C. | C. |
| 10 | Ti/Sb | 8.4 | 4.5 | 12.9 | 0.66 | 10.7 | 5.1 | 6.7 |
| 11 | Ge | 8.7 | 4.6 | 13.3 | 0.68 | 9.5 | 5.1 | 8.4 |
| 12 | Ti/Sb | 6.4 | 4.6 | 11.0 | 0.66 | 6.1 | 2.6 | 3.3 |
| 13 | Ge | 6.7 | 4.8 | 11.5 | 0.65 | 4.9 | 2.5 | 3.7 |

### Examples 14-17: Crystallization Half-Times for Copolyesters with 12 Mole % Total Glycol Modification

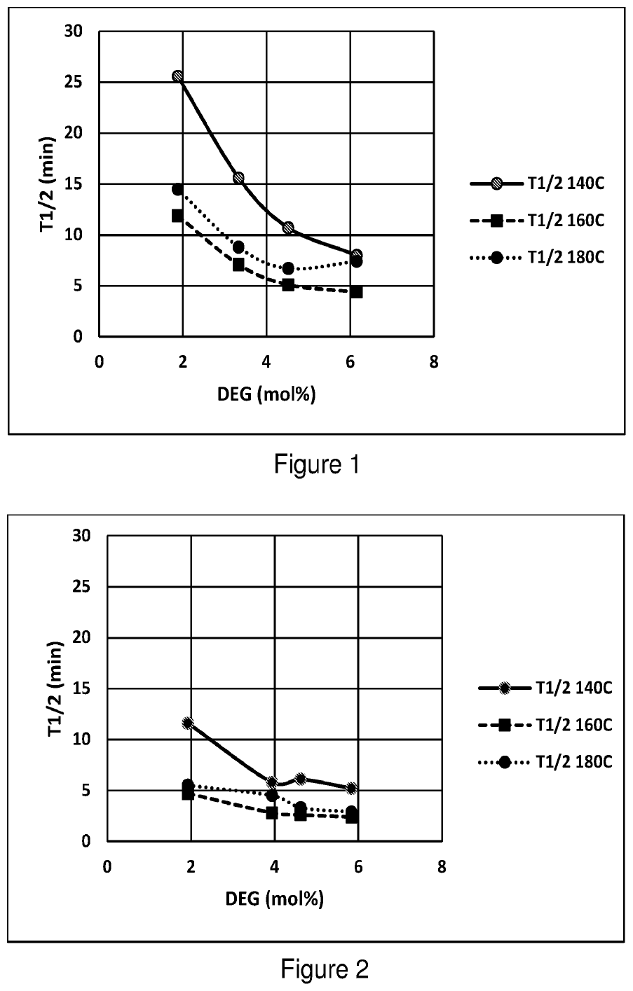

Copolyesters similar in molecular weight with about a 12 mole % total glycol modification were obtained using the procedures described in Examples 1-4 and the results are provided in FIG. 1. In all cases the crystallization half-time was greater than 1 minute.

### Examples 18-21: Crystallization Half-Times for Copolyesters with 10 Mole % Total Glycol Modification

Copolyesters similar in molecular weight with about a 10 mole % total glycol modification were obtained using the procedures described in Examples 1-4 and the results are provided in FIG. 2. In all cases the crystallization half-time was greater than 1 minute.

That which is claimed is:

1. A copolyester composition comprising at least one copolyester and at least one polymeric component; wherein said copolyester comprises:
a. terephthalate acid residues;
b. about 85 to about 96 mole % of ethylene glycol residues;
c. about 4 to about 15 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD); and
d. a germanium catalyst present in the copolyester at a concentration of about 5 to about 500 ppm based on elemental germanium; wherein the terephthalate monomer is based on the substantially equal diacid equivalents of 100 mole % to diol equivalence of 100 mole % for a total of 200 mole %.

2. The copolyester composition according to claim 1 wherein said polymeric component is selected from the group consisting of nylon; polyamides; polystyrene; polystyrene copolymers; styrene acrylonitrile copolymers; acrylonitrile butadiene styrene copolymers; poly(methylmethacrylate); acrylic copolymers; poly(ether-imides); polyphenylene oxides; poly(phenylene oxide)/polystyrene blends; polyphenylene sulfides; polyphenylene sulfide/sulfones; poly(ester-carbonates); polycarbonates; polysulfones; polysulfone ethers; and poly(ether-ketones) of aromatic dihydroxy compounds; or mixtures of any of the foregoing polymers.

3. The copolyester composition of claim 1 wherein said terephthalic acid residues in said copolyester are from at least one monomer selected from the group consisting of terephthalic acid and dimethyl terephthalate.

4. The copolyester composition of claim 1 wherein said copolyester further comprising a dicarboxylic acid selected from aliphatic dicarboxylic acids having 3 to 12 carbon atoms, cycloaliphatic dicarboxylic acids having 8 to 14 carbon atoms and aromatic dicarboxylic acids having 8 to 16 carbon atoms.

5. The copolyester composition of claim 3 wherein said terephthalic acid residues in said copolyester range from 70 to 100 mole %.

6. The copolyester composition of claim 1 wherein said copolyester further comprising up to 10 mole % of one or more modifying aromatic dicarboxylic acids.

7. The copolyester composition of claim 1 wherein said copolyester further comprising up to 10 mole % of one or more aliphatic dicarboxylic acids containing 2-16 carbon atoms.

8. The copolyester composition of claim 1 wherein said copolyester further comprising at least one additional aliphatic, alicyclic, and aralkyl glycol.

9. The copolyester composition of claim 1 wherein the amount of ethylene glycol residues in said copolyester ranges from about 85 to about 92 mol %.

10. The copolyester composition of claim 1 wherein said copolyester comprises about 4 to about 12 mole % of a combination of diethylene glycol (DEG) residues and at least one glycol residue selected from the group consisting of 1,4-cyclohexanedimethanol residues (CHDM), monopropylene glycol residues (MPG), and 2,2,4,4-tetramethyl-1,3-cyclobutane diol residues (TMCD).

11. The copolyester composition of claim 1 wherein the amount of germanium present in the copolyester is at a concentration of about 5 to about 450 ppm.

12. The copolyester composition according to claim 1 wherein the glycol component of the polyester portion of said copolyester further comprises up to 10 mole % of one or more modifying glycols which are not 2,2,4,4-tetramethyl-1,3-cyclobutanediol, ethylene glycol, diethylene glycol, 1,4-cyclohexanedimethanol, or monopropylene glycol.

13. The copolyester composition according to claim 12 wherein said modifying glycol in said copolyester is at least one selected from the group consisting of 1,2-propanediol, 1,3-propanediol, neopentyl glycol, isosorbide, 1,4-butanediol, 1,5-pentanediol, 1,6-hexanediol, p-xylene glycol, polytetramethylene glycol and mixtures thereof.

14. The copolyester composition according to claim 1 wherein said copolyester further comprising at least one branching monomer.

15. The copolyester composition according to claim 1 wherein said copolyester further comprising at least one chain extender.

16. The copolyester composition according to claim 1 wherein said copolyester is capable of being recycled.

17. The copolyester composition according to claim 1 wherein said copolyester has a crystallization half life of greater than 1 minute at 140° C.

18. The copolyester composition according to claim 1 wherein said copolyester has a crystallization half life of greater than 2 minutes at 140° C.

19. The copolyester composition according to claim 1 wherein said copolyester has a crystallization half life of greater than 3 minutes at 140° C.

20. The copolyester composition according to claim 1 wherein said copolyester has a crystallization half life of greater than 1 minute at 140° C., 160° C., and 180° C.
